SEQUENCE LISTING (1 sequence)

SEQ ID NO: 1 - DNA, 869 bp, Homo sapiens
ggatcccgtaaccgaaatcggttgaaccgaaaccggttagtataaaagcagacattttatgcaccaaaaaagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaatcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataatctaccatggctgatcctgcaggtaccgcggccgc

(19) United States
(12) Patent Application Publication
(10) Pub. No.: US 2022/0275339 A1
(43) Pub. Date: Sep. 1, 2022

(54) HEPATIC PRECURSOR-LIKE CELL LINE, CONSTRUCTION METHODS, AND APPLICATIONS IN THE FIELD OF THE BIOARTIFICIAL LIVERS

(71) Applicants: SHANGHAI CELLIVER BIOTECHNOLOGY CO., LTD., Shanghai (CN); SHANGHAI CRYOWISE MEDICAL TECHNOLOGY CO., LTD., Shanghai (CN)

(72) Inventor: Hexin Yan, Shanghai (CN)

(21) Appl. No.: 17/631,474

(22) PCT Filed: Apr. 24, 2020
(86) PCT No.: PCT/CN2020/086660

§ 371 Date: Jan. 29, 2022

(30) Foreign Application Priority Data
Aug. 5, 2019 (CN) 201910718292.5

(51) Int. Cl. C12N 5/071; C12N 15/86; C12N 9/12

(52) U.S. Cl. CPC C12N 5/0672; C12N 15/86; C12N 9/1276; C12N 2740/15042; C12N 2510/04; C12N 2501/60

(57) ABSTRACT

A method of constructing a hepatic precursor-like cell line is provided. The deposited number of the hepatic precursor-like cell line is CCTCC NO: C2019120. The construction method includes using primary hepatocytes as seed cells to establish the hepatic precursor-like cell line, which combines with controlling the conditions of passage culture, subculture, virus infection, proliferation culture and confluence culture. Moreover, a hepatocyte-specific transcription factor, FOXA3, is expressed in the immortalized cell line to make the hepatic precursor-like cell line differentiate easily. An application of the hepatic precursor-like cell line in the field of the bioartificial liver is also provided.

### BACKGROUND

### Technical Field

The present invention relates to the field of the biotechnology, particularly relates to a hepatic precursor-like cell line, a construction method, and applications in the field of the bioartificial livers.

### Description of Related Art

Liver failure is the end stage manifestation of severe liver disease, and the fatality rate of the patients can be as high as 50% to 90%. Liver transplantation is an effective method for the treatment of liver failure. However, because of the high expense, lack of donors, serious immune rejection, and other problems, liver transplantation is difficult to carry out extensively. Currently, there is certainly progress and technological breakthroughs have been made in the research of hepatocyte transplantation and bioartificial liver technology, which is expected to become an effective treatment for liver failure.

The Chinese invention patent application with the publication number CN105521482A discloses the combined application of the different types of the hepatocyte-specific transcription factors, such as HNF1α, HNF4α or FOXP3 to induce differentiation of the hepatocellular carcinoma cells. The patent reveals the above-mentioned hepatocyte-specific transcription factors in the differentiation therapy of liver cancer have good application prospects.

The Chinese invention patent application with the publication number CN103981147A discloses a method for preparing hepatocyte-like cells. This method induces differentiated somatic cells into functional hepatocytes in vitro by overexpressing several transcription factors in somatic cells such as fibroblasts. However, the seed cells of the method are derived from non-hepatic tissues, so only through the reprogramming does not guarantee that it can completely achieve hepatic differentiation, which increases the culture cost of inducing differentiation.

As a result, it is necessary to develop a new type of hepatic precursor-like cell line and its construction to avoid the above-mentioned problems in the prior art.

### SUMMARY

The purpose of this invention is to provide a hepatic precursor-like cell line that can easily accomplish good liver differentiation, a construction method thereof, and an application in the field of bioartificial liver, so as to reduce the cost of culture.

In order to carry out the above purposes, the method for constructing the hepatic precursor-like cell line of this invention comprises:

S1: providing primary hepatocyte cultures, proliferation—and passage-culturing the primary hepatocyte cultures with a passage ratio of 1:3 to 1:6 and a passage number of 2 to 30 to obtain to-be-infected cultures;

S2: confluence-culturing the to-be-infected cultures at a seeding density of 0.5×10⁴-1×10⁵ cells/cm² to obtain adherent cells, wherein the confluence rate of the adherent cells is not less than 80%;

S3: virus-infecting the adherent cells with a lentivirus suspension and a viral infection enhancement buffer, and replacing a medium during the virus infection, wherein the amount of the lentivirus in the suspension is 0.5 to 50 times the adherent cells, and the concentration of the virus infection enhancement buffer is 6-12 μg/ml;

S4: proliferation-culturing and subculturing the infected cells with a selecting agent and an amplifying medium for a proliferation duration of 24-96 hours to obtain immortalized cells with a subculture ratio of 1:3 to 1:6, and a passage number of 10 to 100;

S5: overexpressing a hepatocyte-specific transcription factor in the immortalized cell line to obtain the hepatic precursor-like cell line.

The present invention also provides a hepatic precursor-like cell line prepared by the construction method. The hepatic precursor-like cell line is a viable sample deposited in the China Center for Type Culture Collection in Wuhan University, No. 299 Bayi Road, Wuchang District, Wuhan Hubei Province, China. The preservation number is CCTCC NO.: C2019120, and the classification is named ALI-CELL-85F.

Specifically, the culture is received by the China Center for Type Culture Collection on Jun. 11, 2019 and is named as immortal human hepatic progenitor cell-like cell line ALI-CELL-85F. The culture is tested and identified as a viable state on Jun. 24, 2019. The immortal human hepatic progenitor cell-like cell line ALI-CELL-81.5 is the immortal hepatic progenitor cell-like cell line having a category name of ALI-CELL-85F according to this invention.

The China Center for Type Culture Collection shall, upon request, store the culture for 30 years starting from Jun. 11, 2019, and shall continue to store it for another 5 years after receiving the request to provide culture samples before the expiration date.

The beneficial effect of the construction method of this invention is that since the primary hepatocyte cultures are derived from liver tissues, the primary hepatocytes in the primary hepatocyte cultures are used as seed cells to construct the hepatic precursor-like cell line, which combines with controlling the technical conditions of the passage, the subculture, the virus infection, the proliferation culture, and the confluence culture, as well as overexpresses the hepatocyte-specific transcription factor in the infected cells to obtain the hepatic precursor-like cells line which is easily achieve good liver differentiation.

Preferably, in the step S5, the hepatocyte-specific transcription factor is Forkhead Box A3, FOXA3.

Preferably, in the step S1, a collagenase perfusion method is used to separate the primary hepatocyte cultures from the normal liver tissues.

Preferably, in the step S1, the primary hepatocyte cultures are proliferation-cultured at a seeding density of 0.5×10⁴-2×10⁴ cells/cm² for 6 to 12 days. The beneficial effect thereof is that controlling an appropriate seeding density is helpful to promote the growth in a shorter time and reproduction of the primary cells and creates good conditions for the subsequent subculture.

Further preferably, after the coating buffer is coated in the culture dish with a coating density of 0.87-1.74 μl/cm², the primary cell cultures are seeded in the culture dish, and then the TEM medium is used for the proliferation culture.

Preferably, in the step S2, the to-be-infected cells are confluence-cultured with the coating buffer coated in the culture dish at a coating density of 0.87-1.74 μl/cm² for at least 24 hours. The beneficial effect is that controlling the coating density of the coating buffer is helpful to enhance the adherence ability of the to-be-infected cells to be reached a good confluence rate in a short time.

Further preferably, in the step S3, the lentivirus suspension and the viral infection enhancement buffer are added into the culture dish with the adherent cells, and after 6 to 12 hours, the coating buffer is replaced. The beneficial effect is beneficial to promote the effect of the virus infection further.

Further preferably, after the coating solution is replaced, the adherent cells are continuously cultured for 24 to 72 hours to complete the virus infection.

Further preferably, cultures obtained by the virus infection are performed a fluorescent analysis to count the fluorescent rate, when the fluorescent rate is higher than 90%, the step S4 is performed. The beneficial effect is to ensure the cell obtained after virus infection with a high infection efficiency, which makes for creating the good conditions for the subsequent construction of the immortalized cell line.

Further preferably, the expression gene of the lentivirus is SV40 large T antigen, HPV E6E7 gene or human telomerase reverse transcriptase gene (hTERT gene), and the viral infection enhancement buffer is polybrene buffer. The beneficial effect is that it can effectively improve the efficiency of the virus infection.

Preferably, the cultures obtained by the virus infection and the selecting agent are transferred to a medium to perform the proliferation culture, and the volume ratio of the selecting agent and the medium is 1:1000. The beneficial effect is that the infected cells can be further selected by the selecting agent, which makes for creating the good conditions for the subsequent construction of the immortalized cell line.

Further preferably, the selecting agent is puromycin, hygromycin, or neomycin sulfate, and the medium is TEM medium.

### BRIEF DESCRIPTION OF THE DRAWINGS

FIG. 1 is a schematic diagram of the morphology of the hepatic precursor-like cell line obtained by the first construction method of this invention.

FIG. 2 is a comparison diagram of the gene expression levels of the different functional genes of the control cell line and the first target cell line of this invention.

FIG. 3 is a comparison diagram of the ammonia clearance capacity of the control cell line and the first target cell line of this invention.

FIG. 4 is a comparison diagram of the α1-antitrypsin level of the control cell line and the first target cell line of this invention.

FIG. 5 is a comparison diagram of the Western blotting of the control cell line and the second target cell line of this invention.

FIG. 6 is a comparison diagram of the urea production level of the control cell line and the third target cell line of this invention.

FIG. 7 is a comparison diagram of the albumin level of the control cell line and the third target cell line of this invention.

FIG. 8 is a comparison diagram of the proliferation performance of the control cell line and the first target cell line of this invention.

FIG. 9 is a comparison diagram of the doubling time of the control cell line and the first target cell line of this invention.

FIG. 10 is a schematic diagram of the structure of the bioartificial liver reactor of this invention.

FIG. 11 is a schematic diagram of the proliferation ability of the hepatocytes seeded on the surface of the blank carriers at different culture times during the gas-liquid interactive culture of the first target cell line of this invention in the bioartificial liver reactor shown in FIG. 10.

FIG. 12 is a schematic diagram of the gene expression level of the different functional genes of the hepatocytes seeded on the surface of the blank carriers this invention after the first target cell line of this invention has been cultured for 12 days in the bioartificial liver reactor shown in FIG. 10.

### DETAILED DESCRIPTION

To make the objectives, technical solutions and advantages of this invention clearer, the technical solutions in the embodiments of this invention will be described clearly and completely with reference to the accompanying drawings of this invention. Obviously, the described embodiments are part of, but not all of, the embodiments of this invention. Based on the embodiments of this invention, all other embodiments obtained by those skilled in the art without creative work shall fall within the protection scope of this invention. Unless otherwise defined, the technical or scientific terms used herein shall have the usual meanings understood by those skilled in the art related to this invention. As used herein, “comprising” and other similar terms mean that the elements or objects appearing before the term encompass the elements or objects listed after the term and their equivalents, without excluding other elements or objects.

The main source of the reagents in the embodiments are as follows:

The TEM culture medium, cell suspension of SV40 large T antigen, lentivirus expressing HPV E6E7, and lentivirus expressing the gene of human telomerase reverse transcriptase, hTERT, are from Shanghai Celliver Biotechnology Co., Ltd. The matrigel is produced by Corning Incorporated, and the catalog number is 356234. The polybrene buffer and the neomycin sulfate are produced by Shanghai Yisheng Bio-Technology Co., Ltd, and the catalog numbers are 40804ES79 and 60207ES25 separately. The puromycin is produced by Shanghai Beyotime Biotechnology Co., Ltd., and the catalog number is ST551. FOXA3 is from Shanghai Genechen Co., Ltd. A hygromycin is produced by Thermo Fisher Scientific (Invitrogen), and the catalog number is 10687010.

The sources of the main instruments in embodiments are follows:

Six-well plates are produced by NEST Science Co., Ltd., and the catalog number is 703001. A cell culture incubator is purchased from ESCO Micro Pte. Ltd. Singapore, the catalog number is NO CLL-170B-8. A cell inverted microscope is purchased from Nikon Co., Ltd., and the catalog number is NO Ta2-FL.

In view of the problems in the prior art, embodiments of this invention provides a constructing method of a hepatic precursor-like cell line, including:

S1: providing primary hepatocyte cultures, proliferation-culturing and passage-culturing the primary hepatocyte cultures with a passage ratio of 1:3 to 1:6 and a passage number of 2 to 30 to obtain to-be-infected cultures;

S2, confluence-culturing the to-be-infected cultures at a seeding density of 0.5×10⁴-1×10⁵ cells/cm² to obtain adherent cells, wherein the confluence rate of the adherent cells is not less than 80%;

S3, virus-infecting the adherent cells with a lentivirus suspension and a viral infection enhancement buffer, and replacing a medium during the virus infection, wherein the amount of the lentivirus in the suspension is 0.5 to 50 times the adherent cells, and the concentration of the virus infection enhancement buffer is 6-12 μg/ml;

S4, proliferation-culturing and subculturing the infected cell with a selecting agent and an amplifying medium for 24-96 hours to obtain immortalized cells with a subculture ratio of 1:3 to 1:6 and a passage number of 10 to 100;

S5, overexpressing a hepatocyte-specific transcription factor in the immortalized cell line to obtain the hepatic precursor-like cell line.

Wherein, the coating density is defined as the volume of the coating buffer per unit culture area.

The seeding density is defined as the number of cells per unit area.

The cultures in the same group of the culture dish are separated and reseeded to another culture dish for the subsequent subculture. The number ratio of the pre-separated cultures to the separated cultures is defined as the passage ratio.

In step S5 of some embodiments of this invention, the hepatocyte-specific transcription factor is FOXA3, the sequence thereof is referred to the sequence listing.

In other embodiments of this invention, the hepatocyte-specific transcription factor is HNF1α or HNG4α.

In step S1 of some embodiments of this invention, the primary hepatocyte cultures are human primary hepatocyte cultures.

In step S1 of other embodiments of this invention, the primary human hepatocytes are separated from the liver tissues of the normal donor or the normal liver tissues adjacent to hemangioma by a collagenase perfusion method. The collagenase perfusion method and the fluorescence-activated cell sorting are described in the “Expansion and Differentiation of Human Hepatocyte-derived Liver Progenitor-like Cells and Their Use for the Study of Hepatotropic Pathogens” and will not be repeated here.

In step S1 of some embodiments of this invention, after coating the coating buffer with a coating density of 0.87-1.74 μl/cm² in the culture dish, the primary cells are seeded in the culture dish at a density of 0.5×10⁴-2×10⁴ cells/cm² to perform the proliferation culture for 6-12 days.

In step S2 of some embodiments of this invention, the to-be-infected cells are confluence-cultured with the coating buffer coated in the culture dish with a coating density of 0.87-1.74 μl/cm² for at least 24 hours.

In step S3 of some embodiments of this invention, the lentivirus suspension and the viral infection enhancement buffer are added into the culture dish with the adherent cells, and after 6 to 12 hours, the medium is replaced. After replacing the medium, the adherent cells are continuously cultured for 24 to 72 hours to complete the virus infection.

In step S4 of some embodiments of this invention, the cultures obtained by the virus infection and the selecting agent are mixed and then transferred to an amplifying medium to perform the proliferation culture, and the volume ratio of the selecting agent and the amplifying medium is 1:1000.

In some specific embodiments of this invention, the coating buffer is referred to as Matrigel. The proliferation culture dish and the confluence culture dish are six-well dishes. The medium is the TEM medium. The expressing genes of the lentivirus are SV large T antigen, HPV E6/7 gene, or hTERT. The viral infection enhancement buffer is polybrene buffer. The selecting agent is puromycin, hygromycin, or neomycin sulfate.

The construction method and the advantages of the hepatic precursor-like cell line are described in detail with embodiments 1-3 as follows Embodiment 1

The embodiment provides a first construction method.

The six-well culture dishes were used in the embodiment with the same specification, and the Matrigel was added to the six-well culture dish in advance. The coating density of the Matrigel was 0.87 μl/cm².

The incubator was used in the embodiment with a constant temperature of 37° C., saturated humidity, and a CO2 concentration of 5%.

In the first construction method, except for the process of culturing the cells in the incubator, other operations were carried out in a laminar airflow bench at room temperature.

Specifically, the first construction method is:

S11: the human primary hepatocytes were seeded in the first culture dish at a density of 1×10⁴ cells/cm². After 2 ml of the TEM medium to each well of the first culture dish was added, the cells were cultured in the incubator for 6 days. As a result, the primary cells in the primary cultures differentiated gradually to obtain the proliferation ability.

Specifically, the human primary hepatocytes were discarded surgical specimens from Shanghai Eastern Hepatobiliary Surgery Hospital. The patients' consent was obtained and the informed consent forms were signed before the operation.

S12: After completing the proliferation culture, the medium was removed from the first culture dish. The cell culture obtained by the proliferation culture was subcultured with a trypsin digestion solution and a TEM medium with a passage number of 20 and a passage ratio of 1:3 to obtain the to-be-infected cells.

Specifically, 0.5 ml of trypsin digestion solution and 2 ml of the cultures obtained by the proliferation culture were added to each well of the second culture dish, and the second culture dish was put into the incubator for 3 minutes to perform a digestion-resuspension treatment. After the digestion-resuspension treatment were completed, 2 ml of TEM medium were added to each well of the second culture dish, and the second culture dish was put into the incubator to be cultured. After the bottoms of the second culture dish were observed to be overgrown with the cells, the cells were divided into 3 parts equally. Moreover, 0.5 ml of trypsin digestion solution was added to the other 3 wells to perform the digestion-resuspension treatment for completing a passage. In the process of the passage, 2 ml of TEM medium in each well was replaced with a new TEM medium every 24 hours to improve the passage efficiency.

S21: After the passage was completed, the to-be-infected cells were seeded in the third culture dish at a density of 2×10⁴ cells/cm². The third culture dish was put into an incubator to perform confluence culture. As soon as the confluence rate reached 80% in the third culture dish was observed, the confluence culture was stopped.

S31: The confluence-cultured cells as mentioned in step S13 were digested and re-suspended with trypsin digestion solution to get the digested cultures. The digested cultures were seeded in the fourth culture dish at a density of 2×10⁴ cells/cm², and the fourth culture dish was put into an incubator. After 24 hours later, the supernatant of the fourth culture dish was removed. Then, the TEM medium, SV large T antigen, and polybrene buffer were added into the fourth culture dish, and the cultures were infected for 8 hours in the incubator. After the virus infection was completed, the TEM medium in each well of the fourth culture dish was replaced, and the fourth culture dish was put into the incubator for continuously culturing. After incubating for 72 hours, a small amount of the cell suspension was taken to be analyzed under the fluorescence-activated microscope. If the fluorescence rate was greater than 90%, the virus infection was completed.

Specifically, 50 μl of the SV large T antigen suspension and 10 μl of the polybrene buffer were added to each well of the fourth culture dish. The ratio of the SV large T antigen's number and the adherent cells' number in the suspension was 20:1. The concentration of the polybrene buffer was 8 μg/ml.

S41: After the virus infection completed, the infected cells were added to the fifth culture dish with the TEM medium and puromycin, and then the fifth culture dish was put into the incubator lasting 24 hours for the proliferation culture.

After the proliferation culture was completed, the TEM medium in the fifth culture dish was replaced with a new TEM medium. Then, the cells in the fifth culture dish were subcultured 20 times with a passage ratio was 1:3 to obtain an immortalized cell line.

Specifically, before the proliferation culture, cells in each well of the fifth culture dish were 0.5 ml, the volume ratio of puromycin and the TEM medium was 1:1000, and the TEM medium was 2 ml.

One-run specific subculture process was as follows: As soon as the pre-subcultured cells were observed to be overgrown at the bottoms of the culture wells, the pre-subcultured cells were separated into 3 parts. Then the cells were subcultured into the other 3 wells, and 2 ml of a TEM medium was added to each well of the culture dish.

S51: FOXA3 was overexpressed in the immortalized cell line to obtain the viable hepatic precursor-like cell line, which was deposited in the China Center for Type Culture Collection. The preservation number was CCTCC NO. C2019120, and the classification was named ALI-CELL-85F.

Specifically speaking, the implementation of the overexpression was mentioned in “Expansion and Differentiation of Human Hepatocyte-derived Liver Progenitor-like Cells and Their Use for the Study of Hepatotropic Pathogens” published in Cell Research, 2018, 29, 1, and will not be repeated here.

### Embodiment 2

The embodiment provides a second construction method. The difference between the second construction method and the first construction method in Embodiment 1 are:

The coating density of Matrigel was 1.74 μl/cm².

In step S11, the seeding density of the human primary hepatocytes was 2×10⁴ cells/cm².

In step S12, the number of passages was 5, the passage ratio was 1:4.

In step S21, the seeding density of the to-be-infected cells was 1×10⁵ cells/cm².

In step S31, the ratio of the lentivirus and adherent cells in the suspension was 50:1. Infection completed, the cells were cultured in the incubator for 48 hours.

In step S41, a TEM medium and hygromycin were added into the fifth culture dish. Moreover, the proliferation culturing was lasted 2 days with a subculture ratio of 1:4 and a subculture number of 40.

### Embodiment 3

The embodiment provides a third construction method. The difference between the third construction method and the first construction method in Example 1 are:

The density of Matrigel was 1 μl/cm².

In step S11, the human primary hepatocytes were proliferation-cultured at a seeding density of 0.5×10⁴ cells/cm² for 3 days.

In step S12, the number of passage was 10, and the passages ratio was 1:6.

In step S21, the seeding density of the to-be-infected cells was 1.5×10⁴ cells/cm².

In step S31, the lentivirus suspension was expressed the hTERT gene. The density of the digested cells was 1×10⁴ cells/cm², and the virus infection lasted 10 hours. The ratio of the lentivirus and the adherent cells was 40:1, and the concentration of polybrene buffer was 10 μg/ml.

In step S41, TEM medium and neomycin sulfate were added to the fifth culture dish. Moreover, the proliferation culturing was lasted 4 days with a subculture ratio of 1:6 and a subculture number of 30.

The Embodiments 1-3 of this invention provide the first, the second and the third construction methods, which had good experimental reproducibility.

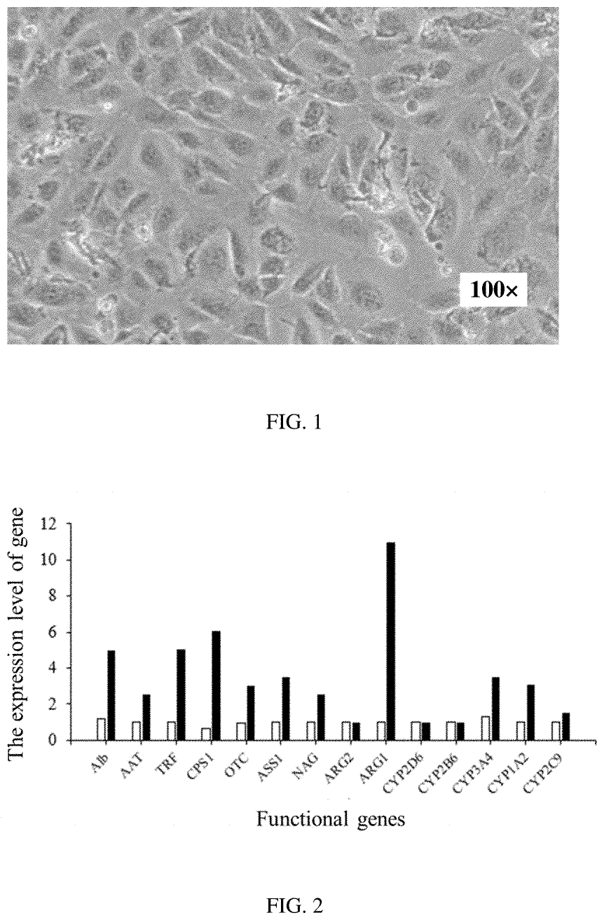

The structure diagram of the morphology of the hepatic precursor-like cell line made by the first construction and observed under a cell inverted microscope at a magnification of 100 times was shown in FIG. 1. The concrete sample preparation and observation methods were ordinary skills in the art and will not be repeated here.

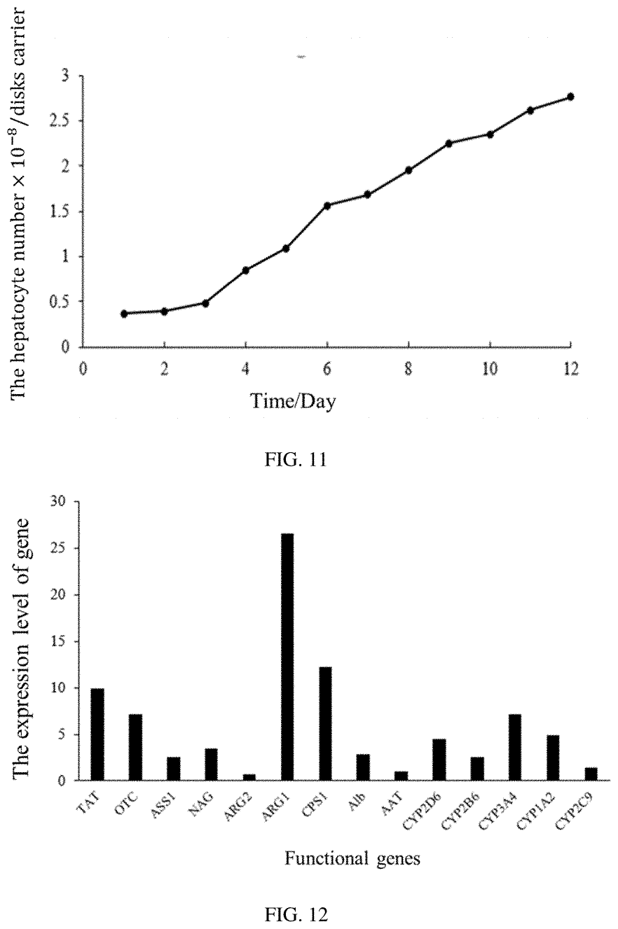

Referring to FIG. 11, the hepatic precursor-like cell line has a more regular morphological structure, which helps to exert favorable cell function.

The infected cell line constructed by the first construction method was used as the control cell line. The hepatic precursor-like cell line constructed by the first construction method was used as the first target cell line. The hepatic precursor-like cell line constructed by the second construction method was used as the second target cell line. The hepatic precursor-like cell line constructed by the third construction method was used as the third target cell line. The gene expression level, protein level, urea production capacity, ammonia clearance capacity, albumin synthesis capacity, and synthesis capacity of α1-antitrypsin (Alpha-1-antitrypsin, AAT) of different functional genes in the control cell line and each target cell line were analyzed and characterized.

Specifically, the RNAfast200 kit, produced by Shanghai Flytech Technology Co., Ltd. and catalog number 220010, was used to respectively extract the RNA of the control cell line and the first target cell line. Then, the RNA was reverse transcribed into cDNA by a reverse transcriptase, which was produced by Invitrogen Corporation, and the catalog number was 18064014. Finally, the relevantly functional genes of hepatocytes were expressed by fluorescence quantitative PCR to obtain a comparison diagram of the gene expression levels of the different functional genes of the control cell line and the first target cell line, shown in FIG. 2. The specific expression method of the fluorescent quantitative PCR was ordinary skills in the art and will not be repeated here.

The related functional genes were Albumin (Alb), AAT, Transferrin (TRF), Carbamoyl phosphate synthetase 1 (CPS1), Ornithine transcarbamylase (OTC), Arginino succinate synthetase 1 (ASS1), Arginase-1 (ARG1), Arginase-2 (ARG2), N-acetyl-beta-D Glucosaminidase (NAG) and 5 drug metabolism enzymes of phase I. The 5 drug metabolism enzymes of phase I were CTP3A4, CYP2D6, CYP2B6, CYP1A2, and CYP2C9.

Referring to FIG. 2, the gene levels of Alb, AAT, and TAT in the first target cell line were higher than the related gene levels in the control cell line. The result was provided that the first target cell line was overexpressed by FOAX3 with the excellent protein synthesis function. The gene levels of CPS1, OTC, ASS1, NAG, and ARG1 in the first target cell line were higher than the related gene levels in the control cell line, while the gene level of ARG2 was basically as same as the control cell line. The result was provided that the urea production capacity of the first target cell line was better than the control cell line. Therefore, the superior urea production capacity indicates that the first target cell line has the excellent detoxification function. The gene levels of CYP3A4, CYP1A2, and CYP2C9 in the first target cell line were higher than the related gene levels in the control cell line, while the gene level of CYP2D6 and CYP2B6 have the little difference with the gene level in the control cell line. The result was provided that the drug metabolism ability of the first target cell line was better than the control cell line.

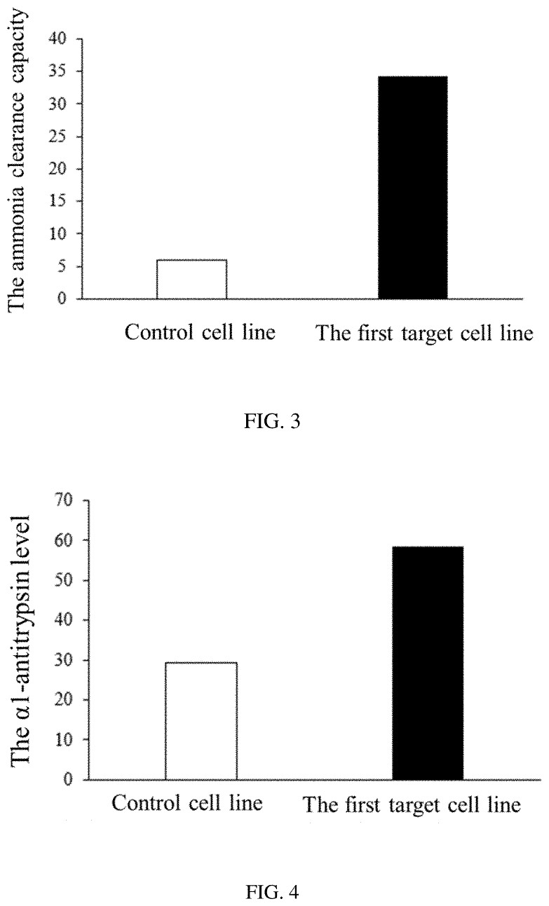

Further on, a rapid kit was produced by Megazyme Incorporation with the product number, K-AMIAR07/14. The rapid kit was used to characterize the ammonia clearance capacity of the first target cell line and the control cell line respectively. A comparison diagram of the ammonia clearance capacity was obtained and shown in FIG. 3.

Referring to FIG. 3, the physical meaning of the ammonia clearance level was the ammonia consumed by per 10⁶ cells per day. The ammonia clearance level of the control cell line was about 5.9, while the ammonia clearance level of the first target cell line was as high as 34.4, which was 6 times the ammonia clearance of the control cell line. As a result, the first target cell line possesses an excellent detoxification function.

Furthermore, an AAT ELISA kit was produced by Bethyl Incorporation with product number, E88-122. The AAT ELISA kit was used to detect the α1-antitrypsin level of the first target cell line and the control cell line respectively. A comparison diagram of the α1-antitrypsin level was obtained and shown in FIG. 4.

Referring to FIG. 4, the physical meaning of α1-antitrypsin level was the nanogram mass of ATT synthesized by per 10⁶ cells per day. The α1-antitrypsin level of the control cell line was 29.2, while the α1-antitrypsin level of the first target cell line was as high as 58.5, which was 2 times the α1-antitrypsin level of the control cell line. The result was provided that the first target cell line possesses the good ATT synthesis capacity.

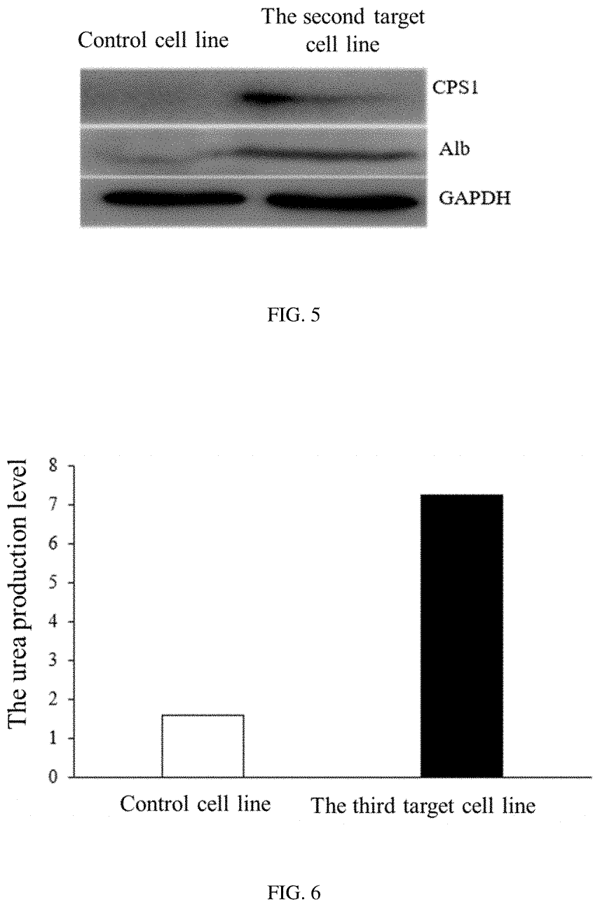

Taken Glyceraldehyde-3-phosphate dehydrogenase (GAPDH) as a reference, the protein level characterization of Alb and CPS1 of the second target cell line and the control cell line were measured by Western blot. Then, a comparison diagram of the protein levels was obtained and shown in FIG. 5.

To be more specifically, the specific implementation of the Western blotting method was mentioned in “Cell Aggregation Culture Induces Functional Differentiation of Induced Hepatocyte-like Cells through Activation of Hippo Signaling” published in Cell Report, 2018, Vol. 25, 1.

Referring to FIG. 5, the GAPDH Western blotting of the second target cell line and the control cell line were not much different. By comparison with the GAPDH Western blotting, the Alb Western blot of the second target cell line was more significant than the Alb Western blot of the control cell line. The result was provided that the second target cell line has a better albumin synthesis function. Moreover, the CPS1 Western blotting of the second target cell line was more significant than the CPS1 Western blotting of the control cell line. The result was provided that the second target cell line has better urea production capacity than the control cell line.

A QuantiChrom™ Urea Assay Kit was produced by Bioassay System Incorporation with the catalog number, DIUR-500. The QuantiChrom™ Urea Assay Kit was used to characterize the urea production capacity of the third target cell line and the target cell line respectively. Then, a comparison diagram of the urea production levels was obtained and shown in FIG. 6. In FIG. 6, the physical meaning of the urea production level was the milligram of the urea produced by per 10⁶ cells per day.

Referring to FIG. 6, the urea production level of the control cell line was about 1.6, while the urea production level of the third target cell line was as much as 7.3, which was 5 times the urea production level of the control cell line. As a result, the third target cell line has excellent detoxification.

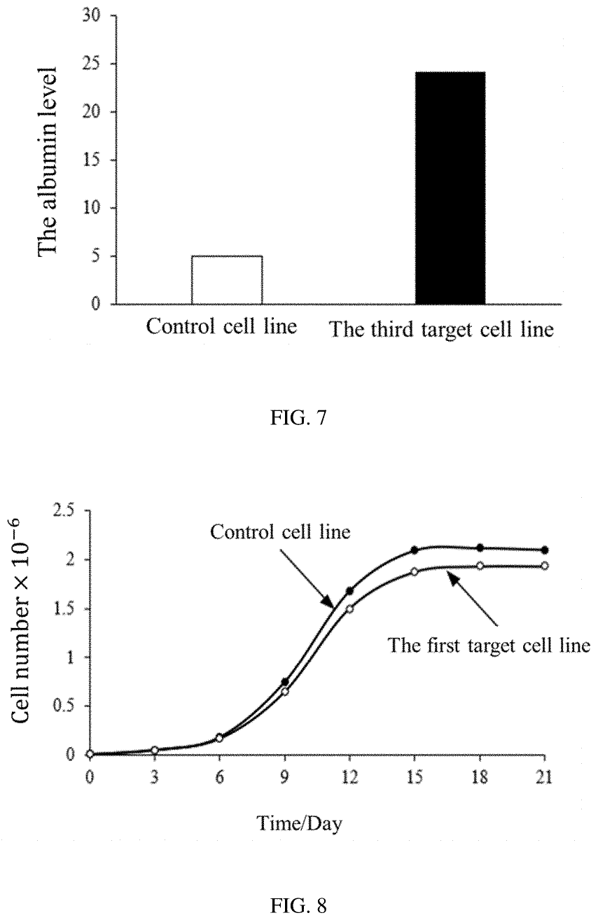

Further on, a human albumin ELISA kit was produced by Bethyl Incorporation with the product number, E88-129. The human albumin ELISA kit was used to detect the album levels of the third target cell line and the control cell line respectively. Then, a comparison diagram of the albumin level was obtained and shown in FIG. 7.

Referring to FIG. 7, the physical meaning of albumin level was the nanogram of Alb produced by per 10⁶ cells per day. The albumin level of the control cell line was about 5.0, while the albumin level of the third cell line was as high as 24.2, which was 5 times the albumin level of the control cell line. Therefore, the result was provided that the third target cell line has the superior albumin synthesis function.

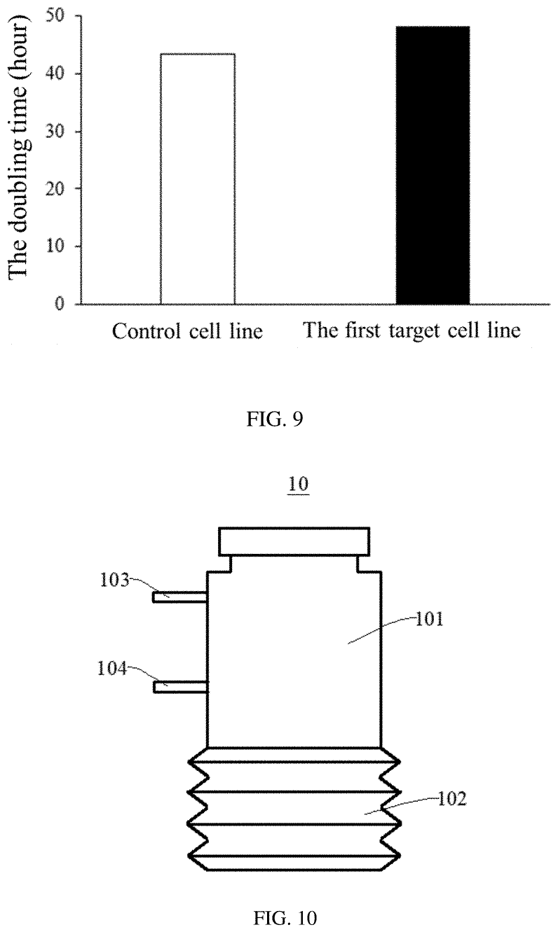

In the embodiments of this invention, the proliferation performance of the first target cell line and the control cell line were characterized. A comparison diagram of the proliferation performance shown in FIG. 8, and the comparison diagram of the doubling time shown in FIG. 9 were obtained.

Specifically, the specific characterized method of the proliferation performance was mentioned in the “Expansion and Differentiation of Human Hepatocyte-derived Liver Progenitor-like Cells and Their Use for the Study of Hepatotropic Pathogens” published in Cell Research, 2018, 29, 1.

Referring to FIG. 8, during the 21 days proliferation and growth period, the number of cells proliferated by the first target cell line and the control cell line and the growth rate were not with an obvious difference. Referring to FIG. 9, the doubling time of the first target cell line was about 48 hours, which was not much different from the doubling time of the control cell line of 43 hours. As a consequence, the growth characteristics of the first target cell line were affected by the overexpression of FOXA3 basically.

Also, the hepatic precursor-like cell line obtained by the construction methods was applied to the field of the bioartificial liver as an embodiment of this invention.

First, a bioartificial liver reactor was provided, referring to Embodiment 2 of the Chinese invention patent application number, CN106620916A, which seeding the hepatocytes on blank disks carriers. Specifically, the difference between the hepatocytes seeded process of CN106620916A and the Embodiment 2 disclosed was that the hepatocytes used in this application were constructed from the first construction method in Embodiment 1, that is, the first target cell line. The mass of the blank disk carriers was 11 g, and the hepatocyte number seeded per gram of the blank disk carriers was 1×10⁶ cells.

Subsequently, the hepatocytes seeded in the disk carriers were subjected to gas-liquid interactive cultured by the bioartificial liver reactor for 12 days, which induces the proliferation and growth of the hepatocytes.

FIG. 10 was a schematic diagram of the structure of the bioartificial liver reactor in some embodiments of this invention.

Referring to FIG. 10, the bioartificial liver reactor 10 includes a container body 101 and a corrugated pipe 102, as well as a liquid inlet pipe 103 and a liquid outlet pipe 104 penetrating the container body 101. The top of the container body 101 was provided with an air vent and an antibacterial breathable membrane to ensure corrugated pipe 102 elevated smoothly and block microorganisms in the air.

The bottom of the container body 101 had a gas-liquid channel, which was not marked in the FIG. 10, to deliver 500 ml of the DMEM medium through the liquid inlet pipe 103 into the corrugated pipe 102. The disks carriers seeded with the hepatocytes were sealed in the container body 101, and the liquid inlet pipe 103 and the liquid outlet pipe 104 were closed. The corrugated pipe 102 was driven by an actuating device, which was not marked in the FIG. 10 and set outside of the bioartificial liver reactor 10 to perform elevating movements periodically. Furthermore, the DMEM medium was driven to enter the container body 101 through the gas-liquid channel, so as to perform the gas-liquid interactive culturing to the disk carriers seeded with the hepatocytes.

In a specific period, the disks carriers seeded with the hepatocytes were immersed in the DMEM medium, and the liquid height was maintained unchanged. The liquid phase material exchange between hepatocytes and culture medium was performed for 60 seconds. After the liquid phase material exchange was completed, the liquid height of the DMEM medium was lower, so that the hepatocytes and the oxygen in the air perform a gas phase material exchange.

After the gas-liquid interactive culture was completed, the liquid outlet pipe 104 was opened, and the medium was discharged through the compressed liquid outlet pipe 104 and the corrugated pipe 102.

In the process of the gas-liquid interactive culture, 3 pieces of the disk carriers seeded with the hepatocytes were taken out of the container body 101 every day to detect the number of hepatocytes. Therefore, a schematic diagram of the proliferation curve shown in FIG. 11 was obtained. The ordinate shown in FIG. 11 represents the number of hepatocytes seeded in the disks carriers per unit mass.

Referring to FIG. 11, during the process of the gas-liquid interactive culture for 12 days, the linear relationship between the number of hepatocytes and the culture time was obvious. Moreover, the number of hepatocytes was significant and the growth trend was stable.

The aforementioned corresponding characterization methods to analyze and characterize the gene expression functional genes in the hepatocytes seeded in the disks carriers after completing the gas-liquid interactive culture to obtain FIG. 12, which was a schematic diagram of the gene expression level of the different functional genes.

Referring to FIG. 12, the hepatic precursor-like cell line obtained by the first construction method were acquired the significant expression in the related functional genes, such as Alb, AAT, CPS1, OTC, ASS1, ARG1, ARG2, NAG, CYP3A4, CYP2D6, CYP2B6, CYP1A2, CYP2C9, and Trans-activating Protein (TAT).

The aforementioned corresponding characterization methods to characterize the albumin synthesis capacity, AAT synthesis capacity, urea production capacity, and ammonia clearance capacity of the hepatocytes seeded in the disks carriers after completing the gas-liquid interactive culture were characterized. The results indicates that the mass of 71.7 ng of Alb produced per day, 163.9 ng of α1-antitrypsin produced per day, 69.7 μg of ammonia consumed per day, and 25.2 mg of urea produced per day for every 10⁶ hepatocytes seeded in the disks carriers complete the gas-liquid interactive culture.

As the mentioned analysis above, the hepatic precursor-like cell line obtained by the first construction method acquires good albumin synthesis function, AAT synthesis function, detoxification function, and drug metabolism ability, after the gas-liquid interactive culture in the bioartificial liver reactor 10.

Although the embodiments of this invention are described in detail above, it is obvious to those skilled in the art that various modifications and changes can be made to these embodiments. However, it should be understood that such modifications and changes fall within the scope and spirit of this invention described in the claims. Moreover, this invention described here may have other embodiments and may be implemented or realized in various ways.

1. A constructing method of a hepatic precursor-like cells line, comprising:
S1: providing primary hepatocyte cultures, proliferation—and passage-culturing the primary hepatocyte cultures with a passage ratio of 1:3 to 1:6 and a passage number of 2 to 30 to obtain to-be-infected cultures;
S2: confluence-culturing the to-be-infected cultures at a seeding density of 0.5×10⁴-1×10⁵ cells/cm² to obtain adherent cells, wherein the confluence rate of the adherent cells is not less than 80%;
S3: virus-infecting the adherent cells with a lentivirus suspension and a viral infection enhancement buffer, and replacing a medium during the virus infection, wherein the amount of the lentivirus in the suspension is 0.5 to 50 times the adherent cells, and the concentration of the virus infection enhancement buffer is 6-12 μg/ml;
S4: proliferation-culturing and subculturing the infected cell with a selecting agent and an amplifying medium for a proliferation duration of 24-36 hours to obtain immortalized cells with a subculture ratio of 1:3 to 1:6 and a passage number of 10 to 100; and
S5: overexpressing FOXA3 in the immortalized cell line to obtain the hepatic precursor-like cell line.

2. (canceled)

3. The construction method according to claim 1, wherein, in the step S1, the primary human hepatocytes are separated from the normal liver tissues of the donor by a collagenase perfusion method.

4. The construction method according to claim 3, wherein, in the step S1, the primary human hepatocytes are proliferation-cultured at a seeding density of 0.5×10⁴-2×10⁴ cells/cm² for 6 to 12 days.

5. The construction method according to claim 4, wherein, performing the proliferation-culturing in a culture dish coated with a coating buffer at a coating density of 0.87-1.74 μl/cm² in advance.

6. The construction method according to claim 1, wherein, in the step S2, performing the confluence-culturing to the to-be-infected cells in a culture dish coated with a coating buffer at a coating density of 0.87-1.74 μl/cm² in advance for at least 24 hours.

7. The construction method according to claim 6, wherein, in the step S3, the lentivirus suspension and the viral infection enhancement buffer are added into the culture dish with the adherent cells, and after 6 to 12 hours, the coating buffer is replaced.

8. The construction method according to claim 7, wherein, after replacing the coating buffer, the adherent cells are continuously cultured for 24 to 72 hours to complete the virus infection.

9. The construction method according to claim 8, wherein, fluorescence analysis is performed on the cell culture obtained after virus infection to calculate the fluorescence rate, and the step S4 is performed when the fluorescent rate is higher than 90%.

10. The construction method according to claim 1, wherein, the expression gene of the lentivirus is SV40 large T antigen, HPV E6E7 gene or human telomerase reverse transcriptase gene (hTERT gene), and the viral infection enhancement buffer is polybrene buffer.

11. The construction method according to claim 2, wherein, in the step S4, the cultures obtained by the virus infection and the selecting agent are mixed and then transferred to an amplifying medium to perform the proliferation culturing, and a volume ratio of the selecting agent and the amplifying medium is 1:1000.

12. The construction method according to claim 11, wherein, the selecting agent is puromycin, hygromycin, or neomycin sulfate, and the medium is TEM medium.

13. A hepatic precursor-like cell line prepared by the construction method of claim 1, wherein the hepatic precursor-like cells line is preserved in China Center for Type Culture Collection with a preservation number of CCTCC NO. C2019120 and has a category name of ALI-CELL-85F.

14. An application of the hepatic precursor-like cells line prepared by the construction method of claim 1 in the field of a bioartificial liver.
